(19) United States
(12) Patent Application Publication
(10) Pub. No.: US 2022/0339233 A1
(43) Pub. Date: Oct. 27, 2022

(54) COMPOSITIONS AND METHODS FOR PREVENTING RECURRENCE OF CANCER

(71) Applicant: YALE UNIVERSITY, New Haven, CT (US)

(72) Inventors: YungChi Cheng, Woodbridge, CT (US); Wing Lam, New Haven, CT (US)

(21) Appl. No.: 17/782,914

(22) PCT Filed: Dec. 8, 2020
(86) PCT No.: PCT/US2020/063764

§ 371 Date: Jun. 6, 2022

Related U.S. Application Data
(60) Provisional application No. 62/945,464, filed on Dec. 9, 2019.

(51) Int. Cl. A61K 36/539; A61K 36/484; A61K 36/65; A61K 36/725; A61K 9/00; A61P 35/00; C07K 16/28; A61K 39/395

(52) U.S. Cl. CPC A61K 36/539; A61K 36/484; A61K 36/65; A61K 36/725; A61K 9/0053; A61P 35/00; C07K 16/2818; C07K 16/2827; C07K 16/2896; A61K 39/3955; C07K 2317/21; C07K 2317/24; A61K 2039/505

(57) ABSTRACT

The present disclosure relates the methods and compositions for preventing the recurrence of a cancer in mammals and enhance efficacy of cancer immunotherapies. The compositions used in the method include an herbal extract YIV-906, which comprises herbal extracts of Scutellaria baicalensis (S), Glycyrrhiza uralensis (G), Paeonia lactiflora (P), and Ziziphus jujuba (Z), or β-glucuronidase treated YIV-906 (YIV-906GU).

### CROSS-REFERENCE TO RELATED APPLICATION

This application claims the benefit of priority to U.S. Provisional Patent Application Ser. No. 62/945,464, entitled “COMPOSITIONS AND METHODS FOR PREVENTING RECURRENCE OF CANCER,” filed Dec. 9, 2019, the disclosure of which is incorporated herein by reference in its entirety.

### BACKGROUND OF THE INVENTION

Immune checkpoint blockade therapy is recognized as a breakthrough in cancer treatment. Currently, the U.S. FDA has approved Ipilimumab (anti-CTLA4), Pembrolizumab (anti-PD1), Nivolumab (anti-PD1), and Atezolizumab (anti-PDL1) for the treatment of several types of cancer. The essential mechanism of action of these antibodies is restoring cytotoxic T-cell function through inhibiting the co-inhibitory pathways via interrupting the interactions between CTLA4-CD80/CD86, PD1-PDL1/PDL2. However, not all patients respond to these immunotherapies. These immunotherapies are also dependent on the tumor type. For example, no or low response rates have been found in pancreatic cancer, colon cancer, and liver cancer patients. Therefore, to increase the immunotherapy response rate, specific target-orientated inhibitors for immunosuppression or agonist to stimulate the immune responses are being developed. However, many of these single target-orientated immune enhancers fail in clinical trials. This could be due to the complexity of tumor environments, where the cancer cells are highly heterogeneous and immune cells are composed of many cell types in different developmental stages.

Considering the aforementioned, there is a need in the art to develop multi-target orientated immune enhancers for cancer immunotherapy. The present invention meets this need.

### BRIEF SUMMARY OF THE INVENTION

The disclosure provides for methods of preventing recurrence of cancer in a mammal.

The method includes administering to a mammalian subject an herbal extract containing herbal extracts of Scutellaria baicalensis (S), Glycyrrhiza uralensis (G), Paeonia lactiflora (P), and Ziziphus jujuba (Z), a fraction thereof, or any active chemical present in the herbal extract or the fraction thereof, and/or (b) β-glucuronidase treated YIV-906 (YIV-906GU) or a fraction thereof, or any active chemical present in the YIV-906GU or the fraction thereof. The mammal is further administered an effective amount of at least one immunotherapeutic agent. Suitable immunotherapeutic agents include immune checkpoint inhibitors and antibodies.

### BRIEF DESCRIPTION OF THE DRAWINGS

The following detailed description of specific embodiments of the invention will be better understood when read in conjunction with the appended drawings. For the purpose of illustrating the invention, there are shown in the drawings specific embodiments. It should be understood, however, that the invention is not limited to the precise arrangements and instrumentalities of the embodiments shown in the drawings.

FIGS. 1A-1B illustrate effects of YIV-906 on the anti-tumor activity of anti-PD1 (YIV-906, 500 mg/kg p.o. bid x 7; anti-PD-1 antibody, 200 μg/mouse i.p. qd) against Hepa 1-6 tumor growth of C57BL6 mice. FIG. 1A is a spot plot showing individual tumor growth for each treatment group during days 0 to 14. FIG. 1B is a graph showing average (±SD) tumor growth for each treatment group during days 0 to 20. Tumor sizes at the beginning were about 180 mm³.

FIGS. 2A-2F illustrate the impact of YIV-906 and/or anti-PD1 on macrophages and M1/M2 signature genes expression of Hepa 1-6 tumor. FIG. 2A are images showing immunohistochemistry staining of F4/80 for macrophage infiltration into Hepa 1-6 tumor after 4-days of treatment. FIG. 2B shows quantification of macrophage of tumor sections after 4-day treatments. FIGS. 2C and 2D show MCP1 and iNOS protein expression of Hepa 1-6 tumor after 4-days of treatment. FIG. 2E is a heat map (significantly be up-regulated: red, significantly by down regulated: green) for indicating the mRNA expression determined by RT-qPCR following treatment at day 4. FIG. 2F is a table showing possibility of being in M1 state based on the signature gene expressions shown in FIG. 2E. P values were obtained from T-test analysis.

FIG. 3 illustrates the effects of YIV-906 on the action of IFNγ or IL4 on polarizing bone marrow derived macrophage (BMDMs) into M1 or M2-like macrophage. FIG. 3 shows the heat map for the mRNA expression levels of BMDM following IFNγ or IL14 with or without YIV-906 or YIV-906GU treatment. For each row (gene), up-regulation of mRNA is highlighted as (red) while down-regulation is highlighted as (green). Numbers in the table indicate the relative fold change gene expression for each treatment condition (average of three independent experiments; all gene expressions were normalized to actin). Bone marrow cells were cultured in the presence of murine M-CSF (10 ng/mL) for 7 days, and then cultured in presence with IFNγ 10 ng/mL to induce polarization to M1-like macrophage while M2 like macrophage were induced by IL-4 20 ng/mL for 24h. YIV-906 or YIV-906GU was added at the same time with IFNγ or IL4. The mRNA expression of M1 or M2 related genes were determined by qRT-PCR following treatment at day 8.

FIGS. 4A-4D illustrate the impact of YIV-906GU on the proteins of the IFNγ signaling pathway of BMDMs. FIG. 4A is an histogram showing effect of YIV-906GU on the IFNγ secretion of BMDMs. Bone marrow cells were cultured in the presence of murine M-CSF (10 ng/mL) for 7 days and then YIV-906 was added to the cells for 24 h. IFNγ in the culture medium was detected by ELISA. FIG. 4B shows western blot analysis for the effect of YIV-906GU alone on IFNγ signaling of BMDMs. FIG. 4C shows western blot analysis for the effect of YIV-906GU on IFNγ signaling of BMDMs. FIG. 4D shows western blot analysis for the effect of YIV-906GU on the action of IL4 on IL4 signaling of BMDMs. Bone marrow cells were cultured in the presence of murine M-CSF (10 ng/mL) for 7 days and then IFNγ 10 ng/mL was added to induce polarization to M1-like macrophage while M2 like macrophage were induced by IL-4 20 ng/mL for 24 h with or without YIV-906. Protein expression or phosphorylation was detected with western blotting. Histone H3 was used for normalization of protein loading.

FIGS. 5A-5C illustrate the impact of YIV-906 on the proteins in the IFNγ signaling pathway. FIG. 5A shows western blot analysis for the effect of YIV-906 alone on IFNγ signaling of BMDMs. FIG. 5B shows western blot analysis for the effect of YIV-906 on the action of IFNγ on IFNγ signaling of BMDMs. FIG. 5C shows western blot analysis for the effect of YIV-906 on the action of IL4 on IL4 signaling of BMDMs. Bone marrow cells were cultured in the presence of murine M-CSF (10 ng/mL) for 7 days cultured and then IFNγ 10 ng/mL was added to induce polarization to M1-like macrophage while M2 like macrophage were induced by IL-4 20 ng/mL for 24 h with or without YIV-906. Protein expression or phosphorylation were detected with western blotting. Histone H3 was used for normalization of protein loading

FIG. 6 illustrates the effect of YIV-906 or YIV-906GU on the action of IFNγ to polarize Raw cell 264.7 into M1-like macrophage. YIV-906 or YIV-906GU could potentiate IFNγ to induce MCP1, TNFa and iNOS (M1 related genes). Raw cell 264.7 were cultured in the presence of murine M-CSF (10 ng/mL) for 3 days, and then cultured in presence of IFNγ 10 ng/mL to induce polarization to M1-like macrophage for 24 h. The mRNA expression were determined by RT-qPCR following treatment at day 8.

FIGS. 7A-7B illustrate the effects of YIV-906 and/or anti-PD1 on the PD1 (FIG. 7A) and PDL1 (FIG. 7B) protein expression of Hepa 1-6 tumor. For western blot analysis for the PD1 and PDL1 protein expression of Hepa 1-6 tumor following, 4 days treatment of anti-PD1−/+YIV-906, Beta-actin was used for normalization of protein loading. Each sample was normalized to a master mix sample (MIX) and the loading was duplicated for each gel. T-test P values are shown in the graph.

FIGS. 8A-8C illustrate the effects of YIV-906 and/or anti-PD1 on T cells in BD1 mice and Hepa 1-6 tumor growth in nude mice. FIG. 8A illustrates the effects of YIV-906 and/or anti-PD1 on activated T cell of Hepa 1-6 tumor, as indicated by GranyzmeB and CD3 staining. FIG. 8B illustrates the effects of YIV-906 and/or anti-PD1 on Treg cell of Hepa 1-6 tumor as indicated by CD3+/FOX3P+. Following 4 day treatment, tumor tissues were digested by dispase and subsequently stained with fluorescence labelled antiFOX3P or antiGranyzme B together with CD3(T cells) and CD45(blood cells). Flow cytometer analysis was used to determine the percentage of Treg or GranyzmeB+ve cells of total T cells. FIG. 8C illustrates the effects YIV-906 and/or anti-PD1 on mRNA expression related to T cell of Hepa 1-6 tumor using qRT-PCR.

FIGS. 9A-9C illustrates the effects of YIV-906 on IDO activity in vitro and in vivo. FIG. 9A is a graph illustrating effects of YIV-906, E. coli glucuronidase treated YIV906 (YIV906GU), and its flavonoids on IDO activity of IDO transfected HEK293 cells in culture. HEK293 cells were transfected with mouse IDO expression plasmids and then seeded for culturing overnight. L-tryptophan 125 μM with or without YIV906, YIV906GU or its flavonoids were added to the wells for 24 hr. The concentration of kynurenine of culture medium was measured using colorimetric based assay. Results were normalized to protein concentration in each well. FIG. 9B shows the effect of different treatments on Kynurenine./tryptophan of Hepa 1-6 tumors. FIG. 9C shows the effects of different treatment on monocytic MDSC of Hepa 1-6 tumors. P values from T-test are indicated in FIGS. 9B and 9C.

FIGS. 10A-10B show a Western blot analysis for the IRF3-P protein expression of BMDM (pre-treated with MCSF 20 ng/mL for 7 days) without YIV-906 (FIG. 10A) or YIV-906GU (FIG. 10B) (pre-treated with recombinant E.coli β-glucuronidase to mimic intestine conditions) were added to the cells for another 24 h. Histone 3 was used for normalization of protein loading.

FIG. 10C is a graph showing that IFNβ in the culture medium (48 h) was detected by ELISA assays.

FIG. 11 illustrates the effect of YIV-906 or YIV-906GU (pre-treated with, E.coli glucuronidase) on CD73 enzyme activity. Recombinant human CD73 enzyme was used in present of AMP (100 μM) as substrate with or without YIV-906 or YIV-906GU for 2 hours. The formation of adenosine was detected by HPLC. Relative area adenosine peaks in present of YIV-906 or YIV-906GU were compared to control.

FIGS. 12A-12C show the effects of various YIV-906 formulations on M1/M2 mRNA expression. FIG. 12A shows the effect of YIV906GU, single herbs (G, P, S and Z: GU treated) or one herb deleted formulation (-G, -P, -S and -Z: GU treated) on the mRNA expression of iNOS/Arg of macrophage. FIG. 12B shows the effect of baicalein, wogonin, chrysin, oroxylin A, and baicalin on the mRNA expression of iNOS/Arg of macrophage. Raw cells were cultured in the presence of murine M-CSF (10 ng/mL) for 3 days, and then cultured in presence with IFNγ 10 ng/mL alone or with YIV-906GU/its components to induce polarization to M1-like macrophage for 24 h. The mRNA expression was determined by RT-qPCR following treatment at day 8. FIG. 12C illustrates the detection of YIV-906 compounds of Hepa 1-6 tumor following oral administration of YIV-906 with or without Anti-PD1 using LC-MS as described herein.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

### Definitions

As used herein, each of the following terms has the meaning associated with it in this section.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, exemplary methods and materials are described.

Generally, the nomenclature used herein and the laboratory procedures in pharmacology, natural product chemistry, and organic chemistry are those well-known and commonly employed in the art.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

As used herein, the term “cancer” is defined as disease characterized by the rapid and uncontrolled growth of aberrant cells. Cancer cells can spread locally or through the bloodstream and lymphatic system to other parts of the body. Examples of various cancers include but are not limited to, bone cancer, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “cure” refers to relieving a subject of a particular disease or disorder, for example, a particular type of cancer.

As used herein, the term “extract” refers to a concentrated preparation or solution of a compound or drug derived from a naturally occurring source, such as an herb or other plant material. Extracts may be prepared by a number of processes, including steeping an herb in solution, or drying and grinding an herb into a powder and dissolving the powder in a solution. An extract may be further concentrated by removing a portion of the solvent after dissolving an amount of the desired compound in the solution. An extract may also be strained or centrifuged to remove any solid material from the solution.

The phrase “inhibit,” as used herein, means to reduce a molecule, a reaction, an interaction, a gene and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein or a gene's stability, expression, function and activity, e.g., antagonists.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof. Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable pharmaceutically acceptable base addition salts of compounds of the disclosure include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N methylglucamine) and procaine. All of these salts may be prepared from the corresponding compound by reacting, for example, the appropriate acid or base with the compound.

The terms “pharmaceutically effective amount” and “effective amount” refer to a non-toxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation. By “pharmaceutical formulation” it is further meant that the carrier, solvent, excipient(s) and/or salt must be compatible with the active ingredient of the formulation (e.g. a compound of the disclosure). It is understood by those of ordinary skill in this art that the terms “pharmaceutical formulation” and “pharmaceutical composition” are generally interchangeable, and they are so used for the purposes of this application.

As used herein, the term “YIV-906” refers to an herbal composition comprising Glycyrrhiza uralensis Fisch (G), Paeonia lactiflora Pall (P), Scutellaria baicalensis Georgi (S), and Ziziphus jujuba Mill (Z). YIV-906 can refer to, for example, to a specific composition comprising S, G, P and Z in a 3:2:2:2 ratio prepared under standard operational procedures, including, in some embodiments, hot water extraction of S, P, G, and Z.

As used herein, the term “prevent,” “prevention,” or “preventing” refers to any method to partially or completely prevent, delay, or slow the onset of one or more symptoms or features of a disease, disorder, and/or condition, for example, cancer. Prevention is causing the clinical symptoms of the disease state not to develop, i.e., inhibiting the onset of disease, in a subject that may be exposed to or predisposed to the disease state, but does not yet experience, or display symptoms of the disease state. Prevention may be administered to a subject who does not exhibit signs of a disease, disorder, and/or condition. In some embodiments, slowing the onset of one or more symptoms or features of a disease or disorder means that, if a recurrence of the disease or disorder or one or more symptoms of the disease or disorder occurs, then the disease or disorder or one or more symptoms of the disease or disorder recur at least about 5%, 10%, 20%, 30%, 40%, 50%, 60%, 70%, 80%, 90%, 95%, or 99% slower than the disease or disorder or one or more symptoms of the disease or disorder would recur in the absence of administering YIV-906 or YIV-906GU.

As used herein, the term “subject,” “patient” or “individual” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

As used herein, the term “therapeutically effective amount” is an amount of a compound of the disclosure, that when administered to a patient, treats, minimizes, and/or ameliorates a symptom of the disease or disorder. The amount of a compound of the disclosure that constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

As used herein, the term “treatment” or “treating” is defined as the application or administration of a therapeutic agent, i.e., a compound useful within the disclosure (alone or in combination with another pharmaceutical agent), to a subject, or application or administration of a therapeutic agent to an isolated tissue or cell line from a subject (e.g., for diagnosis or ex vivo applications), who has cancer, a symptom of cancer or the potential to develop cancer, with the purpose to cure, heal, alleviate, relieve, alter, remedy, ameliorate, improve or affect cancer, the symptoms of cancer or the potential to develop cancer. Such treatments may be specifically tailored or modified, based on knowledge obtained from the field of pharmacogenomics.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual and partial numbers within that range, for example, 1, 2, 2.7, 3, 4, 5, 5.3, and 6. This applies regardless of the breadth of the range.

Additionally, throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The following abbreviations are used herein:
BMDM=bone marrow derived monocytes;
GU=β-glucuronidase;
IFNγ=interferon-gamma;
IL4=interleukin 4;
MDSC=myeloid derived suppressor cell;
STING=stimulator of interferon genes; and
YIV-906GU=β-glucuronidase treated YIV-906 or YIV-906 without glucuronide(s).

The disclosure relates, in one aspect, to the unexpected discovery that the composition comprising herbal extracts YIV-906 or glucuronide conjugated YIV-906 or YIV-906GU (β-glucuronidase treated YIV-906 or YIV-906 without glucuronide) can prevent recurrence of a cancer. In certain embodiments, the herbal extracts, or isolated fractions thereof or active chemicals present therein, can be co-administered to a mammal suffering from cancer in combination with immune checkpoint inhibitors or any other therapeutic agent(s) used for treating cancer to prevent recurrence of the cancer.

Many current immune therapies for cancer attempt to convert “cold tumors” into “hot tumors” so that revived immune cells can attack tumor cells. Immune check point antibodies (inhibitors), such as anti-PD1, anti-PDL1, anti-CTLA4 have led to breakthroughs for the treatment of many tumor types. However, tumor types such as HCC (hepatocellular carcinoma), pancreatic cancer, and colon cancer have had relatively low response rates to these antibodies. Many of these remedies are designed to target a specific target (vs. multiple targets) of the immune cycle. The present disclosure describes that YIV-906 or YIV-906GU, a botanical immunomodulator with a systemic biological effect, can potentiate anti-PD1 action against Hepa 1-6 tumor growth by promoting both adaptive and innate immunity.

With respect to adaptive immunity, it was unexpectedly discovered that YIV-906 in combination with an anti-PD1 agent could significantly decrease PD1 tumor proteins and inhibited PDL-1 expression induced by anti-PD1. Further, YIV-906 can modulate IDO activity and lead to a decrease of MDSC of Hepa 1-6 tumor.

Additionally, IDO inhibitors are reported to enhance the action of anti-PD1, anti-PD-L1, anti-CTLA4 on different types of animal tumors. Many attempts to combine IDO inhibitors with immune check point inhibitors in clinical trials have been made, including epacadostat (IDO inhibitor) and pembrolizumab (ECHO-301/KN-252). However this combination did not show sufficient efficacy in a phase III clinical trial for advanced solid tumors and also had serious adverse effects. This setback has not stopped clinical trials from using IDO inhibitors for the treatment of cancer. For example, BMS-986205 is still being tested in combination with nivolumab as a first or second line therapy for liver cancer [NCT03695250].

Without being bound by theory, a single-target orientated inhibitor, such as an IDO inhibitor alone, may not be sufficiently potent to potentiate anti-tumor activity for immune check point antibodies. In contrast, YIV-906 not only enhances the adaptive immune response but also enhances the innate immune response. With respect to innate immunity, it was unexpectedly discovered that YIV-906 plus anti-PD1 agents could attract more M1 macrophage infiltration, which could be partly due to the induction of MCP1 in the tumors. Interestingly, YIV-906 also increased M1 macrophage tumor infiltration when combined with irinotecan (CPT-11) or sorafenib.

Recently, progress has been made in the understanding of the important role macrophages play in immune check point blockade therapy. There is increasing evidence to support that the presence of M1 macrophages in tumors could enhance the efficacy of chemotherapy and target therapies.

M1 macrophages can kill tumor cells directly by generating NO (nitric oxide) or indirectly by activating T cells. On the other hand M2 macrophages, which have high PD1 expression and low phagocytic activity, promote tumor growth and are not favorable for immunotherapy. Low PD1 expression favors M1 macrophages that have high phagocytic activity and could increase immune check point blockade therapy action. A recent report demonstrated that anti-PD1 agents could help switch macrophage polarity states from the M2 to the M1 phenotype in lung cancer. The use of anti-PD1 agents alone can increase the probability of M1 macrophage in the tumor microenvironment by about 40%. In some embodiments, surprisingly, YIV-906 combined with anti-PD1 agents can further enhance M1 macrophages and the innate immune response in the tumor microenvironment.

In various embodiments, YIV-906 combined with an anti-PD1 agent can even further decrease PD1 proteins in tumor tissues, which can subsequently provide favorable conditions for M1 macrophage proliferation with high tumor phagocytosis. As detailed elsewhere herein, the decrease in PD1 protein levels in the YIV-906 plus anti-PD1 group can also explain, without being bound by theory, how lower dosages (at least about ⅓ compared to anti-PD1 alone), of anti-PD1 combined with YIV-906 can achieve the same anti-tumor activity as higher doses of anti-PD1 agent alone.

Boosting innate and adaptive immunity by increasing M1 macrophages by administering YIV-906, and re-activating adaptive immunity by administering anti-PD1 agents in combination, can have a surprisingly strong synergistic effect against Hepa 1-6 tumor growth in vivo. The combination not only eradicated the Hepa 1-6 tumors in every mouse, it also mimicked tumor-specific vaccine-like behavior as demonstrated by selective rejection of re-implanted Hepa 1-6 tumors and the growth of implanted CMT167 or Pan02 tumors.

IFNγ plays an important role in macrophage M1 polarization. Without being bound by theory, YIV-906 can potentiate the IFNγ activity to increase the signaling transduction response to a higher level; as anti-PD1 alone could activate T cells which released IFNγ in tumor, adding YIV-906 could further amplify the IFNγ signal and enhance M1 macrophage polarization. These M1 macrophages have high levels of iNOS protein for metabolizing L-arginine into citrulline and NO, which can kill cancer cells.

Another surprising property of YIV-906 was the inhibitory activity demonstrated on the M2 inducer, IL4, through the down-regulation of IFR4. When treated with the combination of YIV-906 and anti-PD1, the dual effect of promoting M1 polarity while inhibiting the M2 state ensures the dominance of M1 macrophages in tumor tissues.

In some embodiments, Scutellaria baicalensis (S) can promote M1 macrophage polarization. In some embodiments, one or more flavonoids are the pharmaceutically active compounds in S that promote M1 macrophage polarization. The presence of baicalein, wogonin, and oroxylin A was detected in the Hepa 1-6 tumor and these flavonoids can potentiate IFNγ in the tumor to polarize macrophages into M1. It should be noted that the flavonoids are not simply passing through the intestine and getting into tumor sites. After oral administration, most flavonoids of YIV-906 will be subjected to de-glucuronidation by β-glucuronidase from the gut microbiome, such as E.coli. For instance, baicalin (with glucuronide) will be converted into baicalein (without glucuronide). Aglycone flavonoids will be glucuronidated by different UDP-glucuronosyltransferase (UGT) isozymes to form different metabolites of glucuronidated flavonoids when passing through the intestine. The tumor β-glucuronidase could also convert metabolites of glucuronidated flavonoids into aglycone flavonoids, such as wogonin. The ratio of UGT and β-glucuronidase could affect the presence of glucuronidated flavonoids and convert them into aglycone flavonoids in tumors or other tissues. Tumors in the YIV-906 plus anti-PD1 group had more wogonin and oroxylin A than the YIV-906 group, but did not have baicalein.

### Methods

In one embodiment, the disclosure includes a method of preventing recurrence of a cancer in a mammal, wherein the method comprises administering to the mammal in need thereof a therapeutically effective amount of at least one herbal composition selected from the group consisting of: (a) an herbal extract YIV-906 or a fraction thereof or any active chemical present in the herbal extract or the fraction thereof, (b) glucuronide conjugated

YIV-906 or a fraction thereof, or any active chemical present in glucuronide conjugated YIV-906 or the fraction thereof, (c) YIV-906GU (β-glucuronidase treated YIV-906 or YIV-906 without glucuronide) or a fraction thereof, or any active chemical present in YIV-906GU or the fraction thereof. In certain embodiments, herbal extract YIV-906 comprises herbal extracts of Scutellaria baicalensis (S), Glycyrrhiza uralensis (G), Paeonia lactiflora (P), and Ziziphus jujuba (Z). In certain embodiments, the mammal is further administered at least one least immunotherapeutic agent.

In another embodiment, the disclosure includes a method of slowing recurrence of a cancer in a mammal, wherein the method comprises administering to the mammal in need thereof a therapeutically effective amount of at least one herbal composition described herein and, in certain embodiments, at least one immunotherapeutic agent.

In certain embodiments, the cancer comprises a solid tumor. In certain embodiments, the cancer is at least one selected from the group consisting of melanoma, non-small cell lung cancer, renal cell carcinoma, liver cancer, colon cancer, urothelial bladder cancer, and pancreatic cancer.

In certain embodiments, the at least one immunotherapeutic agent is an immune checkpoint inhibitor selected from the group consisting of an anti-PD1, an anti-PD-L1 and an anti-CTLA4. In certain embodiments, the at least one immune checkpoint inhibitor is selected from the group consisting of Ipilimumab, Pembrolizumab, Nivolumab, Durvalumab, and Atezolizumab.

In certain embodiments, the at least one immunotherapeutic agent is an antibody selected from the group consisting of siglec 15 antibody, anti-phosphatidylserine, anti-OX40, anti-CD73, anti-TIM3, anti-CD24, anti-CD47, anti-PD1, anti-PDL1, anti-CTLA4, anti-GITR, anti-CD27, anti-CD28, anti-CD122, anti-TIGIT, anti-VISTA, anti-ICOS, and anti-LAG3.

In certain embodiments, administering the herbal composition enhances the response of the at least one immunotherapeutic agent.

In certain embodiments, the herbal composition is administered to the mammal orally. In certain embodiments, the herbal composition is administered to the mammal in a form selected from the group consisting of a pill, tablet, capsule, soup, tea, concentrate, dragees, liquids, drops, and gelcaps.

In certain embodiments, the therapeutically effective amount of the herbal composition is about 20 mg/day to about 2000 mg/day. In certain embodiments, the therapeutically effective amount of the herbal composition (YIV-906 or YIV-906GU) is about 20, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or about 2000 mg/day.

In one specific embodiment, the therapeutically effective amount of the herbal composition is, for example, about 1600 mg/day.

In certain embodiments, the herbal composition is administered twice daily. In certain embodiments, the herbal composition is administered for about one about two weeks, followed by a suspension of treatment for at least one week.

In certain embodiments, wherein the herbal composition is administered, twice daily, about 30 mins before administering a chemotherapy or a radiation therapy. In certain embodiments, the administering in continued for about 4 days.

In certain embodiments, the herbal composition is administered at a time selected from prior to, simultaneously with, and after administration of the one or more immunotherapeutic agent to the mammal.

In certain embodiments, administering the composition potentiates IFN y action in polarizing macrophages into M1 (or tumor rejection) phenotype. In certain embodiments, administering the composition inhibits IL4 action in polarizing macrophages into M2 (or tumor promotion) phenotype. In certain embodiments, administering the composition promotes STING agonist action. In certain embodiments, administering the composition reduces or inhibits CD73 activity. In certain embodiment, administering the composition has inhibitory effect on indoleamine 2, 3-dioxygenase (IDO) activity.

In certain embodiments, the mammal is a human.

### Administration/Dosage/Formulations

The regimen of administration may affect what constitutes an effective amount. The therapeutic formulations may be administered to the subject either prior to or after the onset of disease or disorder contemplated in the disclosure. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, preferably a mammal, more preferably a human, may be carried out using known procedures, at dosages and for periods of time effective to treat a disease or disorder contemplated in the disclosure. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the disclosure. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 1 and 1,000 mg/kg of body weight/per day. The pharmaceutical compositions useful for practicing the disclosure may be administered to deliver a dose of from 1 ng/kg/day and 100 mg/kg/day. One of ordinary skill in the art would be able to study the relevant factors and make the determination regarding the effective amount of the therapeutic compound without undue experimentation.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the disclosure.

In certain embodiments, the compositions of the disclosure are formulated using one or more pharmaceutically acceptable excipients or carriers. In other embodiments, the pharmaceutical compositions of the disclosure comprise a therapeutically effective amount of a compound of the disclosure and a pharmaceutically acceptable carrier. In yet other embodiments, the compound of the disclosure is the only biologically active agent (i.e., capable of treating cancer) in the composition. In yet other embodiments, the compound of the disclosure is the only biologically active agent (i.e., capable of treating cancer) in therapeutically effective amounts in the composition.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds and/or compositions of the disclosure for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 400 mg to about 6,000 mg, about 500 mg to about 5,000 mg, about 750 mg to about 4,000 mg, about 1,000 mg to about 3,000 mg, about 1,000 mg to about 2,500 mg, about 20 mg to about 2,000 mg and any and all whole or partial increments therebetween. In certain embodiments, the dose of the compounds and/or compositions of the disclosure is about 800 mg.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder contemplated in the disclosure.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the disclosure include oral nasal, rectal, intravaginal, parenteral, buccal, sublingual, or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-peritoneal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are soups, teas, concentrates, tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation”. For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present disclosure also includes a multi-layer tablet comprising a layer providing for the delayed release of one or more compounds of the disclosure, and a further layer providing for the immediate release of a medication for treatment of a disease or disorder contemplated in the disclosure. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intra-peritoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1, 3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds useful within the methods of the disclosure may be administered in the form of microparticles, for example by injection, or in the form of wafers or discs by implantation.

In one embodiment of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any and all whole or partial increments thereof after drug administration.

### Dosing

The therapeutically effective amount or dose of a compound of the present disclosure depends on the age and weight of the patient, the current medical condition of the patient and the progression of a disease or disorder contemplated in the disclosure. The skilled artisan is able to determine appropriate dosages depending on these and other factors.

A suitable dose of a compound, composition, or extract of the present disclosure can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 5 or more times per day. When multiple dosages are used, the amount of each dosage may be the same or different. For example, a dose of 1 mg per day may be administered as two 0.5 mg doses, with about a 12-hour interval between doses.

In various embodiments, the amount or dose of the YIV-906 or YIV-906GU herbal extract administered can be from about 0.5 mg/kg to about 5000 mg/kg, about 1 mg/kg to about 2500 mg/kg, about 5 mg/kg to about 1000 mg/kg, or about 10 mg/kg to about 1000 mg/kg. In various embodiments, the amount or dose of the YIV-906 or YIV-906GU herbal extract administered can be about 0.01, 0.5, 1, 2, 3, 4, 5,6, 7, 8, 9, 10, 20, 30, 40, 50, 60, 70, 80, 90, 100, 120, 140, 160, 180, 200, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340, 1360, 1380, 1400, 1420, 1440, 1460, 1480, 1500, 1520, 1540, 1560, 1580, 1600, 1620, 1640, 1660, 1680, 1700, 1720, 1740, 1760, 1780, 1800, 1820, 1840, 1860, 1880, 1900, 1920, 1940, 1960, 1980, 2000, 2500, 3000, 3500, 4000, 4500, or about 5000 mg/kg. These amounts of YIV-906 or YIV-906GU herbal extract can be administered using any of the dosing regimens described herein.

In various embodiments, the amount or dose of any immune checkpoint inhibitor or immunotherapeutic agent described herein can be from about 0.01 mg/kg to about 50 mg/kg, about 0.05 mg/kg to about 30 mg/kg, or about 1 mg/kg to about 20 mg/kg. In various embodiments, the amount or dose of any immune checkpoint inhibitor or immunotherapeutic agent described herein can be 0.01, 0.05, 0.1, 0.2, 0.3, 0.4, 0.5, 0.6, 0.7, 0.8, 0.9, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, 10, 10.2, 10.4, 10.6, 10.8, 11, 11.2, 11.4, 11.6, 11.8, 12, 12.2, 12.4, 12.6, 12.8, 13, 13.2, 13.4, 13.6, 13.8, 14, 14.2, 14.4, 14.6, 14.8, 15, 15.2, 15.4, 15.6, 15.8, 16, 16.2, 16.4, 16.6, 16.8, 17, 17.2, 17.4, 17.6, 17.8, 18, 18.2, 18.4, 18.6, 18.8, 19, 19.2, 19.4, 19.6, 19.8, or about 20 mg/kg. In some embodiments, the maximum administered daily amount or dose of any immune checkpoint inhibitor or immunotherapeutic agent described herein can be about 10, 20, 30, 40, 50, 60, 70, 80, 90, 100, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340, 1360, 1380, 1400, 1420, 1440, 1460, 1480, 1500, 1520, 1540, 1560, 1580, 1600, 1620, 1640, 1660, 1680, 1700, 1720, 1740, 1760, 1780, 1800, 1820, 1840, 1860, 1880, 1900, 1920, 1940, 1960, 1980, or about 2000 mg.

In some embodiments, YIV-906 and a single immunotherapeutic agent are the only therapeutically active agents in a pharmaceutical composition. In various embodiments, YIV-906GU and a single immunotherapeutic agent are the only therapeutically active agents in a pharmaceutical composition. In some embodiments, YIV-906 or YIV-906GU and an anti-PD1 checkpoint inhibitor are the only therapeutically active agents in a pharmaceutical composition administered to a subject. In some embodiments, YIV-906 or YIV-906GU and an anti-PD-L1 checkpoint inhibitor are the only therapeutically active agents in a pharmaceutical composition administered to a subject. In some embodiments, YIV-906 or YIV-906GU and an anti-CTLA4 checkpoint inhibitor are the only therapeutically active agents in a pharmaceutical composition administered to a subject. YIV-906 or YIV-906GU can be administered either concurrently or sequentially with any of the immunotherapeutic agents described herein. In some embodiments, a smaller amount of an anti-PD1, anti-PDL1, and/or anti-CTLA4 agent is needed to produce a therapeutic effect when administered with YIV-906 or YIV-906GU as compared to administering the anti-PD1, anti-PDL1, and/or anti-CTLA4 agent alone. The smaller amount of an anti-PD1, anti-PDL1, and/or anti-CTLA4 agent can be a dose that is about 1, 2, 3, 4, 5 6, 7, 8, 9, 10, 11, 12, 13, 14, 15, 16, 17, 18, 19, 20, 25, 30, 33, 35, 40, 45, 50, 55, 60, 65, or about 70% smaller when administered with YIV-906 or YIV-906GU as compared to administering the an anti-PD1, anti-PDL1, and/or anti-CTLA4 agent alone.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 5 or more times per day). When multiple daily doses are used, the unit dosage form may be the same or different for each dose.

Toxicity and therapeutic efficacy of such therapeutic regimens are optionally determined in experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure. Particularly useful techniques for particular embodiments will be discussed in the sections that follow.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, specifically point out the preferred embodiments of the present disclosure, and are not to be construed as limiting in any way the remainder of the disclosure.

### Materials and Methods

### Animal Studies

Hepa 1-6 cells (about 2×10⁶ cells in 100 μL phosphate-buffered saline) were transplanted subcutaneously into 4 to 6 week-old female C57BL6 mice (Charles River Laboratories, Wilmington, Mass.). Body weight, tumor size, and mortality of the mice were monitored daily. After 10-14 days, mice with tumor sizes of 180 mm³ were selected. Tumor volume was examined by using the formula length×width×²×π/6. Each group consisted of seven mice. YIV-906 was administered orally for four days (500 mg/kg po, twice per day), while anti-PD1 was administered intraperitoneally for seven days (200 μg/mouse, once per day). In the control groups, mice were orally administered water. On Day 0, YIV-906 was administered 30 minutes prior to anti-PD1 administration.

In various embodiments, the anti-PD1 agent used in the experiments and figures described herein is a mouse anti-PD1 monoclonal antibody, clone G4, hamster IgG.

### Immunohistochemistry

After 4 days of treatment, mice were terminated by cervical dislocation two days or four days after initiation of the drug treatment. Intestinal and colon tissues were removed, fixed in formalin, embedded in paraffin, and sectioned into 10 μm. The sections were mounted on Superfrost slides, dewaxed with xylene, and gradually hydrated. Antigen retrieval was achieved by 10 mM sodium citrate pH 6.0 with 0.02% Tween-20 under steaming for 30 minutes. The primary antibodies were diluted using Tris-HCl buffer containing 1% BSA and 0.5% Tween-20 and were incubated at room temperature for one hour. As a negative control, a set of slides was processed without primary antibody. Super-picture immunohistochemistry detection kit (Invitrogen, Inc.) was used for detection. The slides were counterstained with hematoxylin and mounted. The antibodies used were: Cleaved Caspase-3(#9664, Cell Signaling Technology, Inc.), Cleaved Caspase-8(#9496, Cell Signaling Technology, Inc. Danvers, Mass.), Cleaved Caspase-9 (#ab52298, Abcam, Cambridge, England), F4/80(#ab16911, Abcam).

### Flow Cytometry Analysis

Tumor tissues (200 mg) were cut into small pieces in 0.5 ml RPM1 640 culture medium. Liberase was added to dissociate the connected tumor cells at room temperature for 15 minutes. Dissociated cells were passed through a cell strainer (70 μm). After spinning down the cells at 1000 g centrifugation for 10 min, red blood cells were lysed with 1 mL BD pharm lyse on ice. Cells were collected at 1000 g centrifugation for 10 min. 2×10⁶ cells were used for each staining sample. Cells were re-suspended in RPM1 640 with 3% FBS. Anti-mouse CD16/CD32 clone 2.4G2 (BD Pharmingen, #553142) was used block Fc receptors on cells. Total T cells were stained by Anti-CD3-PE (BD pharmingen, clone 145-2c11, #553064) for 30 minutes on ice. Fixation/Permeabilized (eBioscience) was used to fix and permeabilize cells. The activated cytotoxic T-cells were further stained with Anti-Granzyme B-pacific blue (BioLegend, clone GB11, #515408) and T regulatory cells were stained with Anti-FOX3P-APC (eBioscience, clone FJK16s, #17-5773-83). The stained cells were washed and analyzed by flow cytometry LSR II (BD Canto II, New Jersey, USA).

### Western Blot

BMDM or RAW264.7 cells (American Type Culture Collection) were cultured RPMI supplemented with 5% FBS in 37° C. incubator with 5% CO₂. 2×10⁶ cells were seeded in 12-well plate. After drug treatment, cells were lysed in 0.3 ml protein loading buffer (for 20 ml buffer, 4 mL 10% SDS, 0.75 mL Tris-HC1 (pH 6.8), 5 mL 10% glycerol, 0.5 mL β-mercaptoethanol, and bromophenol blue) for each well, and sonicated for 30 s to break DNA. The cell extract were electrophoresed through Mini PROTEAN® TGX™ Precast gels (12%, 15 well comb, 15 μL/well Cat. #456-1046) in a running buffer (10×, Tris 30 g, Glysine 144 g, SDS 10 g, with double distilled H₂O) and transferred to the nitrocellulose membrane (Bio-Rad Laboratories, Inc) in a transfer buffer (Tris 30 g, Glysine 144 g, SDS 0.5 g). The membrane was blocked and probed in TBS-T buffer (TBST+1% Tween, AB14330-01000, American Bionanlytical) containing non-fat milk 1:5000 (Blotting-Grade Blocker, Cat. #170-0604 Nonfat dry milk).

Primary antibodies (PD-1 (D7D5W) XP® Rabbit mAb #846515 Mouse Specific lot:1 Ref: 08/2017) at 1:1000 in TBS-T buffer (TBST +1% Tween, AB14330-01000, American Bionanalytical) were incubated with the membrane with shaking overnight at 4° C. Histone H3 was used as an internal control for normalization and was detected with a monoclonal actin antibody diluted at 1:1000 (H3(D1H2) XP® Rabbit mAb #4499S Ref: 06/2017). After washing with TBS-T three times, each time for 5 min, the membranes were then further incubated with goat anti-rabbit IgG-HRP SC-2004, lot #B1711 HRP conjugated 1:5000, and incubated in room temperature for 1 hour. Then the membrane was washed with TBS-T three times again. Stable Peroxide solution 1 mL (SuperSignal™ West Pico PLUS, Prod#1863097) and Luminol/Enhancer solution 1 mL (SuperSignal™ West Pico PLUS, Prod#1863096) were used for visualizing and scanning with densitometer. Antibody list: PD-1 (D7D5W) XP® Rabbit mAb #84651S Mouse Specific lot:1 Ref: 08/2017 (Cell signaling), Anti-PD-L1 antibody [EPR20529]ab213480, Arginase-1(D4E3MTM) XP® Rabbit mAb#93668 (Cell signaling), iNOS Antibody (Mouse Specific) #2982 (Cell signaling), Jak1(6G4)Rabbit mAb#3344 (Cell signaling), P-Jak1 (Y1034/1035)(D7N4Z)Rabbit mAb#74129 (Cell signaling), Jak2(D2E12) XP® Rabbit mAb #3230(Cell signaling), P-Jak2 (Y1008)(D4A8)Rabbit mAb #8082(Cell signaling), Stat1 Antibody#9172 (Cell signaling), Phospho-Stat 1(Tyr701)(D4A7)Rabbit mAb#7649(Cell signaling), Stat2(D9J7L)Rabbit mAb#72604 (Cell signaling), Phospho-Stat2(Tyr690)-R sc-21689 #K1609(SantaCruz), Stat6(D3H4)Rabbit mAb#5397 (Cell signaling), Phospho-Stat6(Tyr641)(D8S9Y)Rabbit mAb#56554 (Cell signaling), IRF-1(D5E4) XP® Rabbit mAb #8478(Cell signaling), IRF-4(D9P5H)Rabbit mAb#15106 (Cell signaling).

Quantitative Real Time PCR (qRT-PCR)

Total RNA was extracted with TRIzol reagent (Invitrogen, California, USA). The aqueous phase was collected and then one volume of ethanol was added, following the manufacturer's instructions. Before centrifuging, this slurry was added to a column (miRNeasy, Qiagen, Venlo, Limburg) for further extraction and simultaneous DNA digestion (RNase-Free DNAse set, Qiagen). cDNA was synthesized using random primers and reverse transcriptase MMLV (New England Biolabs, Ipswich, MA). qPCR assays were performed using iTaq™ SYBR® Green Supermix and the CFX96 Real-Time PCR Detection System (Bio-Rad Laboratories, Hercules, Calif.). Relative expression of target genes against (3-actin was expressed as 2^{-ΔCt} and fold differences was calculated as expressed mRNA of YIV-906 and or anti PD1-treated samples against untreated samples. Primers sequences are showed in Table 1.

TABLE 1
Sequences for primers used in qRT-PCR:

| | SEQ | | |
|---|---|---|---|
| | ID | Forward (F) | |
| Gene | NO: | Reverse (R) | DNA Sequence |
| mIFNg | 1 | F | ggcaaaaggatggtgacatgaaa |
| | 2 | R | tagtaatcaggtgtgattcaatg |
| mIFNb | 3 | F | tgtctttcttgtcttcagaaa |
| | 4 | R | tttctgaagacaagaaagaca |
| mgrB | 5 | F | cactctcgaccctacatggcctt |
| | 6 | R | gtgacatttattatacttccttcac |
| mperf | 7 | F | gtgtcgcatgtacagttttcgcctg |
| | 8 | R | ccgtgataaagtgcgtgccatag |
| mPDL1 | 9 | F | cacttgctacgggcgtttactatca |
| | 10 | R | gaatcacttgctcatcttccttttc |
| mPD1 | 11 | F | gccaggatggttcttagactccccag |
| | 12 | R | taccagtttagcacgaagctctccg |
| mIL17a | 13 | F | tcaccctggactctccaccgcaatg |
| | 14 | R | acagaattcatgtggtggtccagc |
| mCTLA4 | 15 | F | tcactgctgtttctttgagca |
| | 16 | R | ggctgaaattgcttttcacat |
| TIMS | 17 | F | ctactacttgcaaggtcattgg |
| | 18 | R | ccaggtgtagatagagtgtaac |
| ICOS | 19 | F | tcagacttttaacaggagaaatc |
| | 20 | R | tctcagggtatttacaagaaatc |
| dectinl | 21 | F | agtgctgggtgccctagcattttg |
| | 22 | R | aggctgagaaaaacctcctgtagt |
| Oasla | 23 | F | gcctttgatgtcctgggtcatg |
| | 24 | R | ccagcttctccttacacagttg |
| Prkra | 25 | F | gaatcattcatggaaactggaaag |
| | 26 | R | atgtccaaccacgttcgttagag |
| illSra | 27 | F | ggagaggtatgtctgtaac |
| | 28 | R | gaggggtctctgatgcacttga |

### Cytokine Analysis by Cytometric Bead Array

Animal plasma and tumor tissue of YIV-906 and or anti-PD-1-treated mice and control mice were collected after 96 hours following the treatments. Culture medium of untreated and YIV-906-treated BMDMs was collected after 24 hours of exposure. Determination of cytokine expression (IL-6, MIP-la, IL-5, IL-17A, IL-12p70, TNFa, IL-1B, IL-10, MIG, IFNγ, MCP-1, G-CSF) was performed using cytometric bead array flex set kit by flow cytometry (BD Canto II, New Jersey, USA) according to the manufacturer's instructions (BD biosciences, UK).

### Isolation of Bone Marrow Derived Monocytes (BMDMs) and Macrophage Differentiation

Bone marrow cells were collected from tibias and femurs of 10-week-old C57B1/6 mice were cultured with complete RPMI-1640 medium (supplemented with 5% Fetal Bovine Serum and 1% Penn/Strep) in the presence of murine M-CSF (10 ng/mL) for 7 days to allow differentiation of monocytes into macrophages. Macrophage were cultured in 5% FBS RPMI-1640 medium with IFNγ (10 ng/mL) to induce polarization to M1-like macrophage while M2 like macrophage were induced by IL4 (20 ng/mL).

### IDO Activity Assay

2×10⁶ HEK293 cells were transfected with mouse IDO (2 μg/10 cm plate) for 48 h. For one plate, 1 mL PBS was used to collect cells into a 2 ml tube. Cells were centrifuged at 3,500 rpm 1 min. Cells were then sonicated in ice cold PB buffer (1 mL, pH 6.5). Cell lysis was clarified by centrifuging at 12,000 rpm for 5 min at 4° C. 25 μL cell lysis solution was mixed with YIV906 or YIV906GU (25 μL) at desired concentrations. Reaction buffer containing 50 μL PB buffer (100 mM, pH 6.5), 10 μL methylene blue (2.5%), 100 μL catalase (20 mg/mL), 250 μL L-tryptophan (500 mM) and, for every 10 mL of total solution, 70 mg of vitamin C. The reaction buffer was then added to the cell lysis solution. The solution was allowed to react for 1.5 h at 37° C. Trichloroacetic acid 30% (25 μL) was added and incubated at 50° C. for 1 hr. Ehrlich's reagent 0.8% [4-(dimethylamino)benzaldehyde, 80 mg/10 mL in acetic acid, 100 μL, from Sigma Aldrich] was added. Absorbance at 540 nm was measured using a UV-vis spectrometer to determine kynurenine concentration. Absorbance at 540 nm (yellow) has been found to have a positive correlation to the amount of kynurenine in a sample.

### CD73 Activity Assay

The CD73 nucleotidase activity was determined by the formation of adenosine from AMP by CD73 over time. The reaction was carried at 37° C. in 200 μL buffer containing 50 mM Tris-HCl (pH 7), 100 mM NaCl, 1 mM MgCl₂, 1 mM CaCl₂, 100 μg/mL BSA, 10 mM AMP, and 200 ng human recombinant CD73 for 3 h. The reaction was extracted with 15% trichloroacetic acid. The supernatant containing the nucleoside and its phosphorylated forms was extracted with a 45/55 ratio of trioctylamine and 1,1,2-trichlorotrifluoroethane. Adenosine was analyzed by high pressure liquid chromatography (Shimadzu, Braintree, Mass.) using a Partisil SAX column (Whatman, Clifton, N.J.) and 10 mM phosphate buffer as mobile phase.

### LC-MS Detection

Each tumor sample were homogenized in 200 μL acetronitrile/methanol/water(2/2/1, v/v/v) and 1 mm glass beads (BioSpec Products, Bartlesville, Okla.) for 30 s at 3500 rpm twice. The homogenate was then centrifuged at 12000 rpm for 15 min at 4° C. The supernatant was dried down in a Speedvac. The residue of each tumor sample was re-dissolved in 100 μL of acetonitrile, and vortexed at 3000 rpm for 3 min. The solution was then centrifuged at 12000 rpm at 4° C. for 15 min, and 2 μL supernatant was injected into the UPLC-QTOF system for analysis. All sample analyses were performed on an ACQUITY ultra-performance liquid chromatography (UPLC) system coupled with a quadrupole-time of flight (Q-TOF) MS instrument (UPLC Xevo G2-XS QTOF MS, Waters Corp., Milford, Mass., USA) with an electrospray ionization (ESI) source. Separation was carried out on a Waters ACQUITY BEH C18 column (2.1 mm×100 mm id, 1.7 μm) with a guard column (Waters ACQUITY BEH C18 column (2.1 mm×5 mm id, 1.7 μm)).

The mobile phase consisted of acetonitrile (A) and water containing 0.1% formic acid (B) using a gradient elution of 5% A at 0-2 min, 5-10% A at 2-3 min, 10-17% A at 3-10 min, 17-30% A at 10-15 min, 30-40% A at 15-20 min, 40-80% A at 20-25 min, 80% A at 25-30 min, 80-5% A at 30-31 min, and 5% A at 31-35 min. The flow rate was 0.3 mL/min. Mass spectrometry was performed on a Water Xevo G2-XS QTOF. The scan range was from 50 to 1000 Da. For the negative electrospray mode, the capillary voltage and cone voltage were set at 2.5 kV and 60 V, respectively. The desolvation gas was set to 800 L/h at a temperature of 500° C. The cone gas was set to 50 L/h at a temperature of 120° C. Data acquisition was achieved using MS^{E}, and the collision energy was 15-60 V.

### Statistical Analysis

Data were analyzed by one- or two-way analysis of variance (ANOVA) (GraphPad Prism 7), correlation analysis (GraphPad Prism 7) and Student's t test (Microsoft Office Excel). The difference was statistically significant when P<0.05.

### Example 1

### YIV-906 Dnhanced Anti-PD1 Action to Inhibit Hepal-6 Tumor Growth In Vivo and Demonstrated Tumor-Specific Vaccine-Like Effect

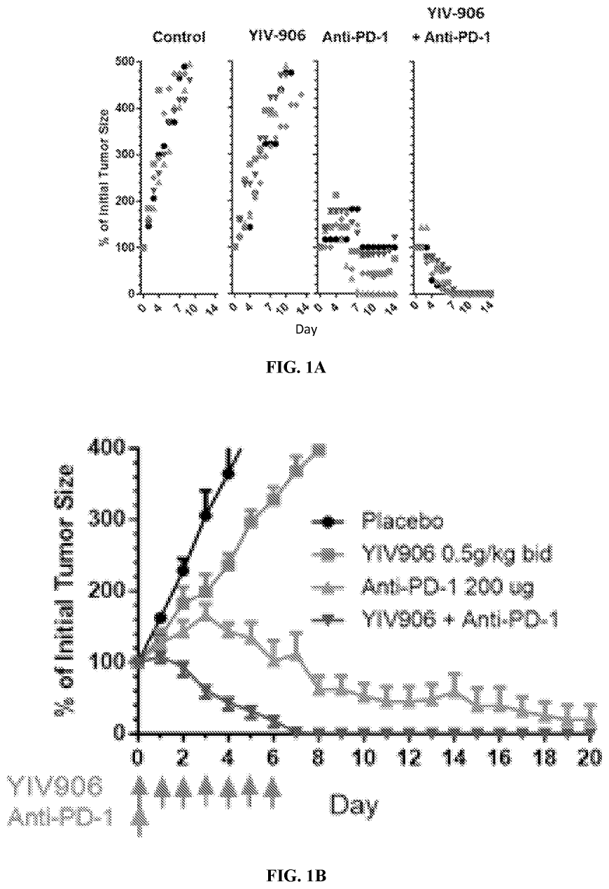

To investigate the effects of YIV-906 and anti-PD1 on Hepa 1-6 tumor growth in NCR nude mice, Hepa 1-6 cells (10⁶ cells) were subcutaneously implanted into NCR nude mice for 10 days. When initial tumor size reached about 180 mm³, YIV-906 (500 mg/kg, p.o.) was administered twice per day from day 0 to day 7 either with or without anti-PD1 (200 μg/mouse i.p. qd) to mice harboring Hepal-6 tumors. Hepal-6 tumor growth was not affected by YIV-906 treatment (P>0.05) (FIGS. 1A and 1B). After 4-days of treatment, anti-PD1 started to slow down tumor growth of Hepa 1-6 (FIGS. 1A and 1B). Some tumor shrinkage was observed on day 8 and by the end of the experiment about 40% tumors were below detection limit (FIGS. 1A and 1B).

The strongest anti-tumor activity was observed in the YIV-906 plus anti-PD1 immune checkpoint inhibitor group. Tumors responded to the combination of YIV-906 and anti-PD1 in as little as 2 days, with all tumors disappearing following 7-days of treatment (P<0.001) (FIGS. 1A and 1B). Without further treatment up to 21 days later, no tumors re-appeared in the YIV-906 plus anti-PD1 combination group. This suggested that the tumors had been prevented from forming and were cured in these mice (FIGS. 1A and 1B). When Hepa 1-6 cells were re-implanted into the cured mice, no tumor growth was found while naïve mice had tumor growth (data not shown). When CMT167 cells (small cell lung carcinoma) or Pan02 cells were implanted into the cured mice after being re-challenged with Hepa 1-6, CMT167, or Pan02, tumor growth was observed. This behavior suggested YIV-906 in combination with anti-PD1 checkpoint inhibitors, or with other immune checkpoint inhibitor therapies, can create a tumor-specific vaccine-like effect to prevent tumor recurrence, in some embodiments. In various embodiments, the combination treatment with YIV 906 and anti-PD1 did not affect the body weight of the mice.

### Example 2

### YIV-906/Anti-PD1 Treatment Induced more Macrophage Infiltration with Higher M1-Like Macrophage Signature in Hepa 1-6 Tumors

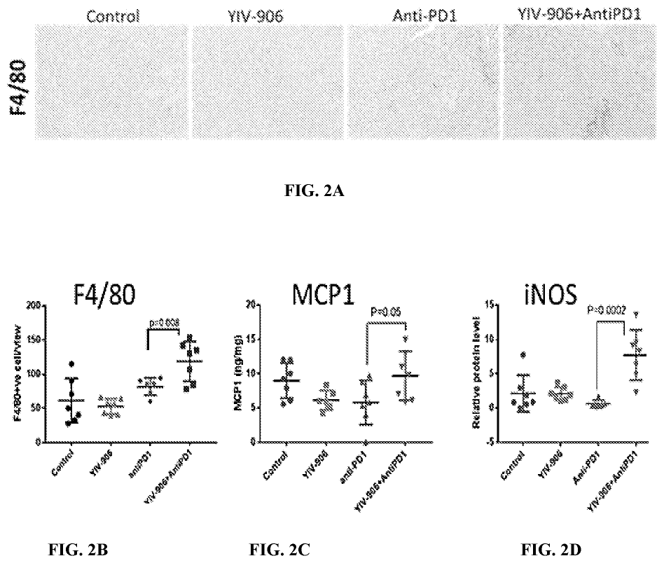

Immunohistochemistry study showed that the combination of YIV-906 and anti-PD1 checkpoint inhibitors, but not YIV906 alone or anti-PD1 alone, significantly induced macrophage infiltration in Hepa 1-6 tumors after 4-days of treatment (FIGS. 2A and 2B). Without being bound by theory, this could be attributed to the increase of MCP1(CCL2), a monocyte chemoattractant protein, of tumors in the YIV-906 plus anti-PD1 treatment group where MCP1 was higher than that of the anti-PD1 only group (P<0.05) (FIG. 2C).

Depending on the tissue microenvironment and which activation pathways' exhibited stimulation, macrophages can be differentiated into two distinct phenotypes: M1 (tumor rejection) and M2 (tumor promotion). Following YIV-906 plus anti-PD1 treatment bio-statistical analysis of the mRNA expression of M1 and M2-like macrophage signature genes suggested that the M1-like macrophages were the dominant phenotype in the tumors (FIGS. 2E and 2F). Western blot analysis further confirmed that the iNOS protein (a M1 marker) was substantially increased following YIV-906 plus anti-PD1 treatment (FIG. 2D). This result also suggested that YIV-906 plus anti-PD1 treated tumors were highly inflamed. Therefore, without being bound by theory, the enhanced infiltration of M1-like macrophages induced by YIV-906 combined with anti-PD1, can be an mechanism aiding against Hepal-6 tumor growth.

### Example 3

### YIV-906 Potentiates IFNγ Action in Polarizing Macrophages into an M1-Phenotype while Inhibiting IL4 Action in Polarizing Macrophages into a M2 Type

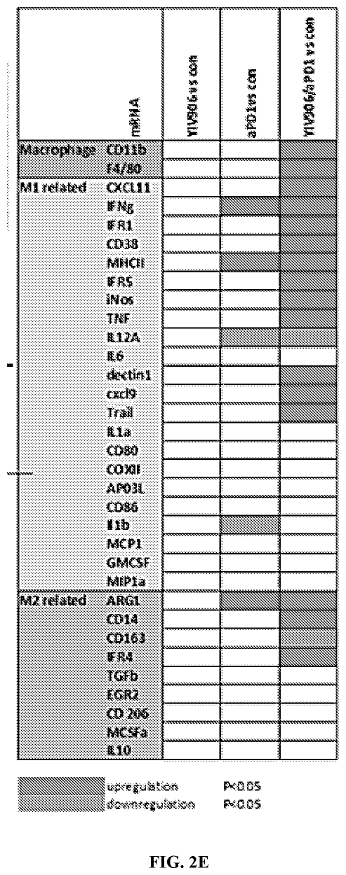

YIV-906 was investigated for any impact on polarizing BMDM into either M1-like or M2-like phenotype in culture. β-glucuronidase (GU) treatment can catalyze hydrolysis of β-D-glucuronic acid residues from certain components of YIV-906, and had effects on the macrophage polarization activity of YIV-906. The results indicated that YIV-906GU had a stronger induction effect on IFNγ, IL1a, TFNα mRNA expression of BMDM than YIV-906 alone (FIG. 3). Furthermore, YIV-906 can potentiate IFN-γ to polarize BMDM into M1 macrophages with increased expression signals of iNOS, MCP-1, CXCL9, CXCL11, COXII, IL1α, TNF-α, and CD86 (FIG. 3). GU treatment further enhanced the potentiation activity of YIV-906 on iNOS, IL1a, CXCL11 (FIG. 3).

Conversely, YIV906 can inhibit the action of IL4 for M2 macrophage polarization exhibited by decreasing mRNA expression levels of Argl, CD206, and IRF4. GU treatment could further increase the inhibitory activity of YIV-906 on Arg, IL10, and IRF4 mRNA expression in the presence of IL4 (FIG. 3). Overall YIV906 can potentiate IFNγ to induce certain M1 associated signature gene expression while inhibiting IL4 to induce certain M2 signature gene expression of BMDM. Without being bound by theory, the immuno-modulatory effect of the above activities could be explained by the sugar moiety of chemicals present in YIV-906, specifically the aglycone chemicals, which appear most active.

### Example 4

### YIV-906 Induces IFNγ Secretion and Activates an Interferon Induction Cascade of BMDM

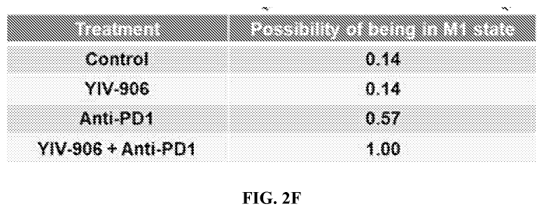

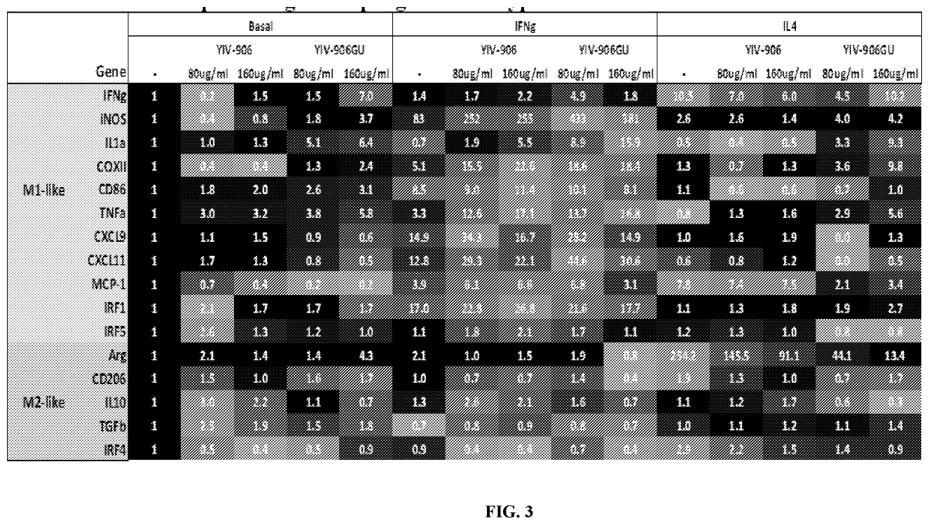

YIV-906 and YIV-906GU (with higher potency) can stimulate IFNγ protein secretion from BMDM (FIGS. 4A-4D). This result showed that YIV-906GU had stronger induction effects on IFNγ mRNA of BMDM (FIG. 3). The increase of IFNγ in the medium triggered the activation of the IFNγ induction cascade as higher P-JAK½, P-stat½, and IRF1 levels were detected under YIV-906GU treatment (FIGS. 4A-4D and FIGS. 5A-5C). The stimulation of IFNβ by YIV-906GU provided an additional mechanism to facilitate M1 macrophage polarization.

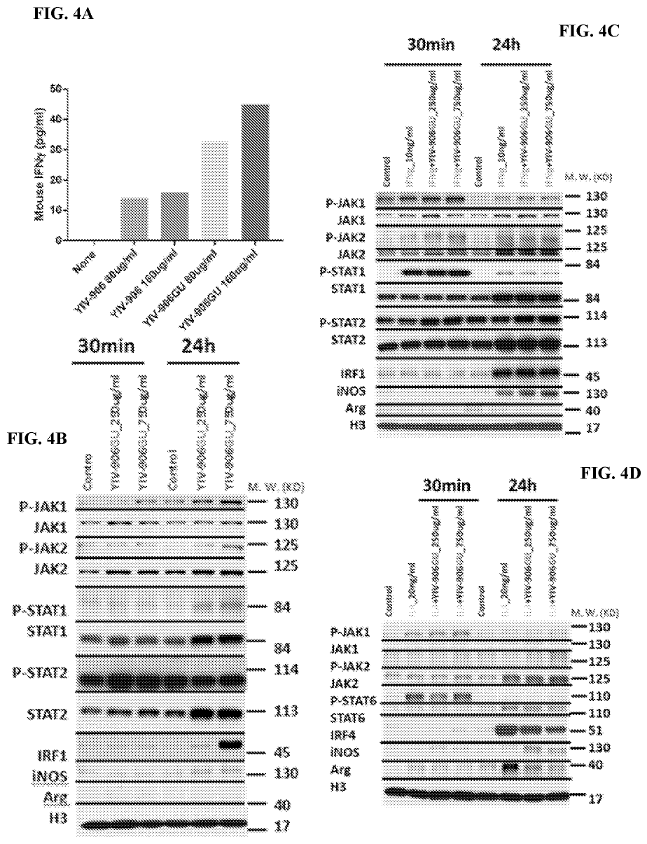

In the presence of IFNγ, YIV-906GU could further enhance P-Jak½ and P-Stat2 protein in as early as 30 min. It could maintain higher P-Stat2 at 24 h in the presence of IFNγ in BMDM. At 24 h YIV-906 or YIV-906GU potentiated IFNγ in inducing iNOS protein expression but not the IFR1 protein of BMDM (FIG. 4A-4d). Without being bound by theory, this could be because IFR1 may have already reached its maximum level at the given concentration of IFNγ. In addition, IL15RA and ICAM mRNA could also be up-regulated by YIV-906GU in the presence of IFNγ in BMDM. YIV-906 or YIV-906GU potentiated IFNγ action is not limited to BMDM, they can also potentiate IFNγ to induce MCP1, TNFa, iNOS mRNA in GM-CSF treated Raw cell 264.7 (macrophages) (FIG. 6).

In contrast to IFNγ, YIV-906, or YIV-906GU inhibited IL4 action by suppressing IRF4 expression, a key transcription factor of the IL4 signaling pathway (FIGS. 4A-4D and FIGS. 5A-5C). Following 24 h treatment of YIV-906 or YIV-906GU, inhibition of IL4 also led to the down-regulation of Arg protein in BMDM (FIGS. 4A-4D and FIGS. 5A-5C). Without being bound by theory, the decrease of IFR4 and Arg protein could be attributed to the down-regulation of their mRNA by YIV-906 or YIV-906GU in the presence of IL4 (FIG. 3).

These results demonstrated that YIV-906 or YIV-906GU themselves could induce IFNγ and IFNIβ secretion. Both can also potentiate IFNγ action by stimulating P-Jak½ and P-Stat2 phosphorylation while inhibiting IL4 action by down-regulating FR4 protein of BMDM. The modality could explain how multiple mechanisms of YIV-906 can work to polarize macrophages into the M1 phenotype favorably.

### Example 5

### Combination of YIV-906 and Anti-PD1 Agents Reduces the PD1 and Normalizes PDL1 Protein Expression of Hepa 1-6 Tumors

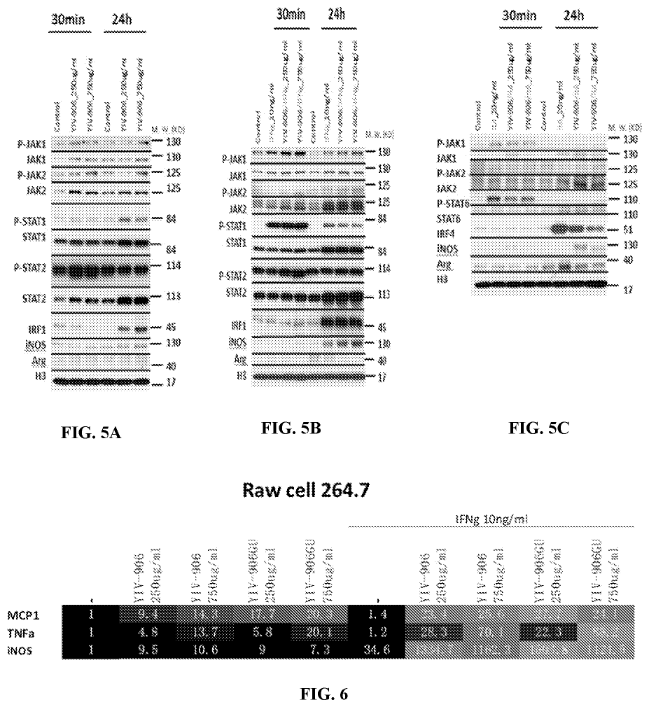

The effects of YIV-906 on the protein expression of PD1 and PDL1 of Hepa 1-6 tumors when combined with anti-PD1 agents were examined. Anti-PD1 or YIV-906 treatment did not significantly change the PD1 tumor proteins. Compared to the control group, YIV-906 plus an anti-PD1 agent can significantly decrease PD1 tumor proteins (P=0.02) or anti-PD1 group (P=0.003) following 4-day treatments (FIG. 7). Without being bound by theory, this result can at least partially explain why less anti-PD1 combined with YIV-906 was required to have similar anti-tumor activity versus a higher dosage of anti-PD1 alone. Additionally, anti-PD1 but not YIV-906-only treatment significantly increased PDL-1 tumor protein (P=0.01), but this increase could be overcome by combining YIV-906 and anti-PD1 (P=0.008) (FIG. 7). Overall these results further suggest that YIV-906 can facilitate the anti-PD1 action in overcoming tumor resistance to immune surveillance.

### Example 6

### YIV-906/anti-PD1 Treatment Induces Gene Expression Related to T cell Activation in Hepa 1-6 Tumors

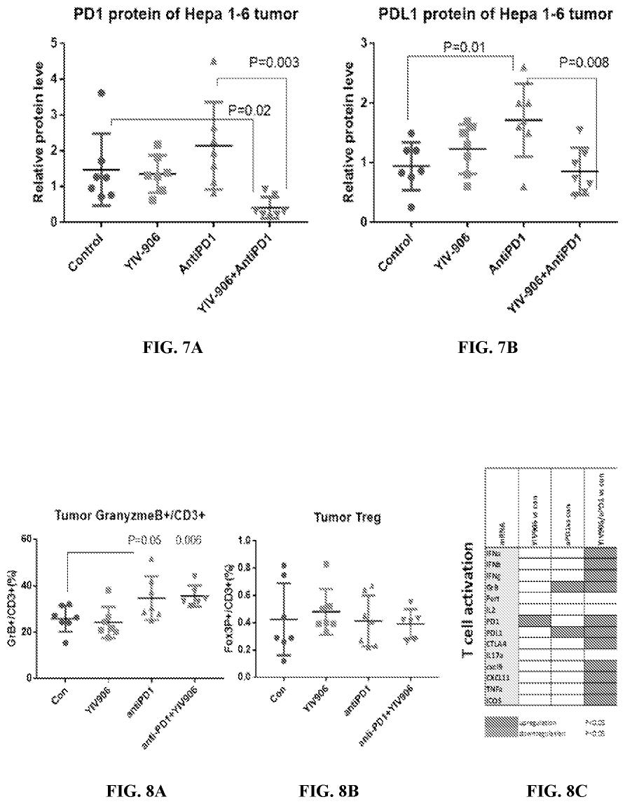

A key function of anti-PD1 is to restore cytotoxic T-cell function by inhibiting the co-inhibitory pathways of T cells. As expected, anti-PD1 agents induced the number of activated T cells (GranyzmeB+/CD3+) of Hepa 1-6 tumors (FIG. 8A). The number of activated T cell and Treg upon anti-PD1 treatment was not affected by the co-treatment of YIV-906 (FIGS. 8A and 8B). However, the combination treatment did induce more T cell activation related genes in Hepa 1-6 tumors (FIG. 8C) and suggests the function of T cells can be enhanced.

The present results indicated that anti-PD1 or YIV-906 monotherapy did not significantly change the PD1 tumor proteins (FIG. 7A). Compared to the control group or anti-PD1 alone group, YIV-906 plus anti-PD1 can significantly decrease PD1 tumor proteins (P=0.02 or 0.003, respectively) following 4-day treatments (FIG. 7A). This result partially helps explain why less anti-PD1 combined with YIV-906 was required to have similar anti-tumor activity versus taking a higher dosage of anti-PD1 alone. Additionally, anti-PD1, but not YIV-906-only treatment, did significantly increase PDL-1 tumor protein (P=0.01) but this increase could be counteracted by combining YIV-906 and anti-PD1 (P=0.008) (FIG. 7B). These results suggested that YIV-906 can facilitate anti-PD1 action in overcoming tumor resistance to immune surveillance and lead to a stronger anti-tumor effect.

### Example 7

### YIV-906 can Modulate Indoleamine 2, 3-Dioxygenase (IDO) Activity, which Plays an Important Role in the Activity of Immune Checkpoint Antibodies

IDO, an enzyme responsible for metabolizing L-tryptophan into kynurenine, can be a key resistance factor to anti-PD1, anti-CTLA4 therapy. IDO inhibitors were reported to enhance the action of anti-PD1, anti-PD-L1, and anti-CTLA4 agents on different types of animal tumors. IDO expression inhibits the activation of effector T cells (Teff) and activation of Foxp3+ regulatory T-cells (Tregs) which help recruit CD11b+Gr1int myeloid derived suppressor cells (MDSCs) into tumors to inhibit T-cell proliferation. Additionally, high monocyte IDO expression was found to favor M2-like macrophage polarization while low expression of IDO in monocytes favors M1-like macrophage polarization.

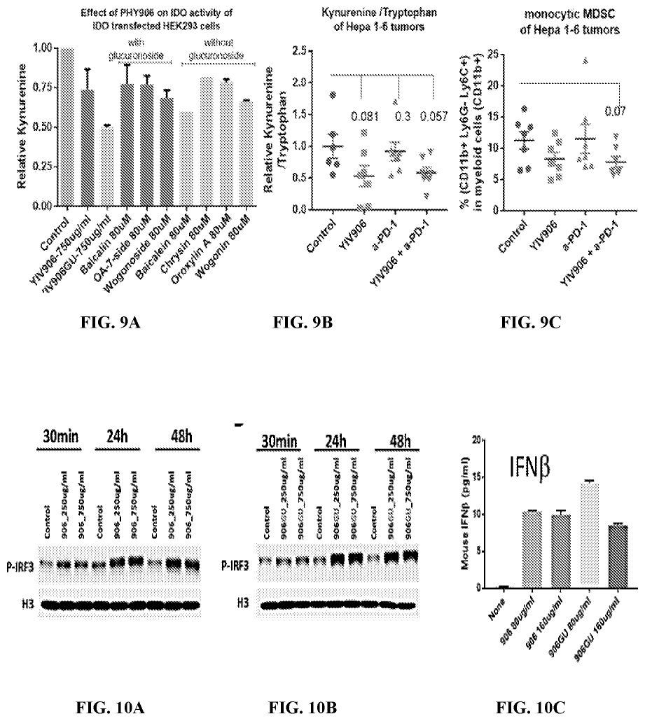

The IDO assay results showed that YIV-906 can modulate IDO enzyme in cell cultures (FIG. 9A). After using purified E.coli glucuronidase (GU) to remove glucuronoside from chemicals to mimic the conditions in lower GI tract, YIV-906GU had stronger IDO inhibition than YIV-906 (FIG. 9A). Baicalein was shown to be the most potent compound among the flavonoids (FIG. 9A). YIV-906 or YIV-90⁶/anti-PD1 had a trend to decrease the kynurenine/tryptophan ratio of Hepa 1-6 tumors (FIG. 9B). This suggested that YIV-906 can modulate IDO activity in vivo. Furthermore, it was found that anti-PD1 plus YIV-906 treatment reduced monocytic MDSC of Hepa 1-6 tumors (FIG. 9C). Modulation of IDO by YIV-906 could be an additional mechanism action to reduce immune tolerance and facilitate the action of anti-PD1.

### Example 8

### YIV906 Increases Phosphorylated IRF3 Protein Levels and IFNβ, which are Key Mediators of STING Signaling

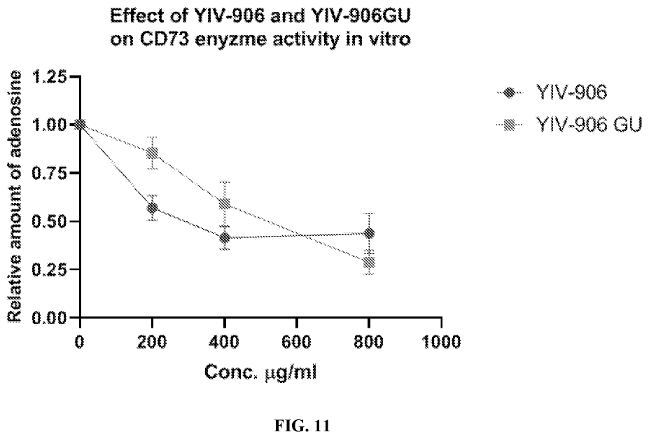

Activation of STING is a recent approach for cancer immunotherapy. STING (stimulator of interferon genes) is a signaling molecule associated with the endoplasmic reticulum (ER) and is important for controlling the transcription of numerous host defense genes. STING signaling can be triggered by cell death double stranded DNA (dsDNA), which binds to cGAS. The dsDNA/cGAS complex will convert ATP and GTP into cGAMP which activates STING to phosphorylate TBK. Finally, phosphorylated TBK will phosphorylate IRF3 for transcription of IFNβ which can activate dendritic cells to recruit and activate T cell against tumors. STING signaling can also play an important role as a tumor vaccine. As shown in FIGS. 10A and 10B, YIV-906 or YIV-906GU (pre-treated with recombinant E.coli β-glucuronidase to mimic intestine conditions) can trigger IRF3 phosphorylation of BMDM (mouse bone marrow derived macrophage). YIV-906 or YIV-906GU treatment (48 h) can also induce IFNβ secretion from BMDM (FIG. 10C).

### Example 9

### YIV-906 Modulates CD73 Enzyme Activity

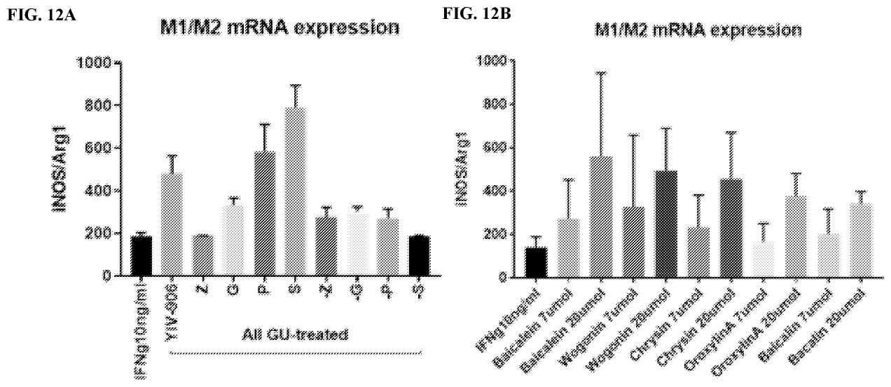

CD73 (5′-nucleotidase (5′-NT) or ecto-5′-nucleotidase) is a membrane nucleotidase responsible to convert extra-cellular AMP into adenosine which bind to A2AR. High levels of extra-cellular adenosine could repress T effector cell function and proliferation by decreasing IL2/IFNγ expression. Adenosine could also inhibit dendrite cells and natural killer cell activities. As shown in FIG. 11, in an in vitro assay, YIV-906 and YIV-906GU inhibited CD73 enzyme activity with various dose inhibition curves. YIV-906 had a stronger inhibitory effect on CD73 than YIV-906GU at 200 μg/mL. YIV-906 could inhibit CD73 by maximum 60% in the range from 400 μg/mL to 800 μg/mL while YIV-906GU had better potency and inhibited CD73 in a dose-dependent manner from 200 μg/mL to 800 μg/mL. These results suggested that glucuronide conjugated compounds of YIV-906 can modulate CD73 activity while aglycone compounds of YIV-906 had a truly inhibitory effect on CD73.

### Example 10

### Flavonoids in YIV-906 Play Important Roles in Potentiating IFNγ Action to Polarize Macrophages into the M1-Like Phenotype

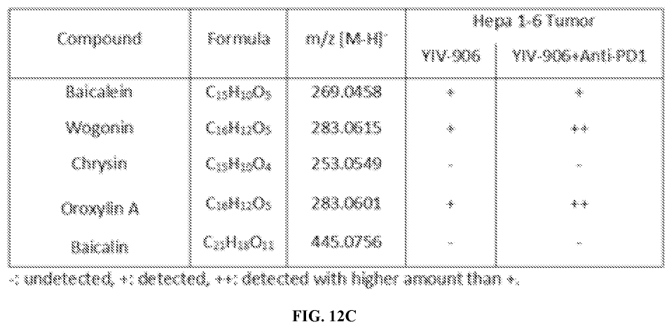

Of the four herb ingredients in YIV-906GU: G, P, S and Z, results indicated that S, in the presence of IFNγ, had the highest biological activity in increasing iNOS/Arg ratio (FIG. 12A). Consistently the formulations without S (-S) lost the IFNγ potentiation property (FIG. 12A). The flavonoids baicalein wogonin, chrysin, oroxylin A, and baicalin are the major marker compounds in S treated with GU and hence subsequently compared the potentiation action on IFNγ. The results indicated that all tested flavonoids could have a positive impact on IFNγ action on increasing iNOS/Arg ratio (FIG. 12B). In some embodiments, deleting any one herb from YIV-906 could reduce the potentiation of IFNγ action (FIG. 12A). These results indicated that G, P, Z could also play a role in the IFNγ potentiation or interact with S to enhance IFNγ action.

An analysis was performed to determine which metabolites of YIV-906 were present in Hepa 1-6 following administration. The results indicated that baicalein, wogonin and oroxylin A, all of which could potentiate IFNγ action (FIG. 12B), were detected in the tumor mass (FIG. 12C). It is important to note that the amount of wogonin and oroxylin A in the tumors was higher in the YIV-906 plus anti-PD1 group compared to the YIV-906 alone group (FIG. 12C). Thus, in some embodiments, these flavonoid compounds present in component S in the YIV-906 and anti-PD1 combination could be the active ingredients, along with others, contributing to the IFNγ potentiation that polarizes macrophages into the M1 phenotype in Hepa 1-6 tumors.

### Enumerated Embodiments

The following enumerated embodiments are provided, the numbering of which is not to be construed as designating levels of importance:

Embodiment 1 provides a method of preventing recurrence of a cancer in a mammal, the method comprising administering to the mammal in need thereof a therapeutically effective amount of at least one herbal composition selected from the group consisting of:
(a) an herbal extract YIV-906 comprising herbal extracts of Scutellaria baicalensis (S), Glycyrrhiza uralensis (G), Paeonia lactiflora (P), and Ziziphus jujuba (Z), a fraction thereof, or any active chemical present in the herbal extract or the fraction thereof, and
(b) β-glucuronidase treated YIV-906 (YIV-906GU) or a fraction thereof, or any active chemical present in the YIV-906GU or the fraction thereof;
wherein the mammal is further administered an effective amount of at least one immunotherapeutic agent.

Embodiment 2 provides the method of embodiment 1, wherein the cancer comprises a solid tumor.

Embodiment 3 provides the method of any one of embodiments 1-2, wherein the cancer is at least one selected from the group consisting of melanoma, non-small cell lung cancer, renal cell carcinoma, liver cancer, colon cancer, urothelial bladder cancer, and pancreatic cancer.

Embodiment 4 provides the method of any one of embodiments 1-3, wherein the at least one immunotherapeutic agent is an immune checkpoint inhibitor selected from the group consisting of an anti-PD1, an anti-PD-L1, and an anti-CTLA4 inhibitor.

Embodiment 5 provides the method of any one of embodiments 1-4, wherein the at least one immune checkpoint inhibitor is selected from the group consisting of Ipilimumab, Pembrolizumab, Nivolumab, Durvalumab, and Atezolizumab.

Embodiment 6 provides the method of any one of embodiments 1-5, wherein the at least one immunotherapeutic agent is an antibody selected from the group consisting of siglec 15 antibody, anti-phosphatidylserine, anti-0X40, anti-CD73, anti-TIM3, anti-CD24, anti-CD47, anti-PD1, anti-PDL1, anti-CTLA4, anti-GITR, anti-CD27, anti-CD28, anti-CD122, anti-TIGIT, anti-VISTA, anti-ICOS, and anti-LAG3.

Embodiment 7 provides the method of any one of embodiments 1-6, wherein administering the herbal composition enhances response of the at least one immunotherapeutic agent.

Embodiment 8 provides the method of any one of embodiments 1-7, wherein the herbal composition is administered to the mammal orally.

Embodiment 9 provides the method of any one of embodiments 1-8, wherein administering the herbal composition promotes stimulator of interferon genes (STING) agonist action.

Embodiment 10 provides the method of any one of embodiments 1-9, wherein the herbal composition is administered to the mammal orally in a form selected from the group consisting of a pill, tablet, capsule, soup, tea, concentrate, dragees, liquids, drops, and gelcaps.

Embodiment 11 provides the method of any one of embodiments 1-10, wherein the therapeutically effective amount of the herbal composition is about 20 mg/day to about 2000 mg/day.

Embodiment 12 provides the method of any one of embodiments 1-11, wherein the therapeutically effective amount of the herbal composition is about 1600 mg/day.

Embodiment 13 provides the method of any one of embodiments 1-12, wherein the herbal composition is administered twice daily.

Embodiment 14 provides the method of any one of embodiments 1-12, wherein the herbal composition is administered for about one to about two weeks, followed by a suspension of treatment for at least one week.

Embodiment 15 provides the method of any one of embodiments 1-14, wherein the herbal composition is administered about 30 mins before administering a chemotherapy or a radiation therapy.

Embodiment 16 provides the method of any one of embodiments 1-14, wherein the administering in continued for about 4 days.

Embodiment 17 provides the method of any one of embodiments 1-16, wherein the herbal composition is administered at a time selected from prior to, simultaneously with, and after administration of the one or more immunotherapeutic agent to the mammal.

Embodiment 18 provides the method of any one of embodiments 1-17, wherein the mammal is a human.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof. The disclosures of each and every patent, patent application, and publication cited herein are hereby incorporated herein by reference in their entirety. While this disclosure is made with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A method of preventing recurrence of a cancer in a mammal, the method comprising administering to the mammal in need thereof a therapeutically effective amount of at least one herbal composition selected from the group consisting of:
(a) an herbal extract YIV-906 comprising herbal extracts of Scutellaria baicalensis (S), Glycyrrhiza uralensis (G), Paeonia lactiflora (P), and Ziziphus jujuba (Z), a fraction thereof, or any active chemical present in the herbal extract or the fraction thereof, and
(b) β-glucuronidase treated YIV-906 (YIV-906GU) or a fraction thereof, or any active chemical present in the YIV-906GU or the fraction thereof;
wherein the mammal is further administered a therapeutically effective amount of at least one immunotherapeutic agent.

2. The method of claim 1, wherein the cancer comprises a solid tumor.

3. The method of claim 1, wherein the cancer is at least one selected from the group consisting of melanoma, non-small cell lung cancer, renal cell carcinoma, liver cancer, colon cancer, urothelial bladder cancer, and pancreatic cancer.

4. The method of claim 1, wherein the at least one immunotherapeutic agent is an immune checkpoint inhibitor selected from the group consisting of an anti-PD1, an anti-PD-L1, and an anti-CTLA4 inhibitor.

5. The method of claim 4, wherein the at least one immune checkpoint inhibitor is selected from the group consisting of Ipilimumab, Pembrolizumab, Nivolumab, Durvalumab, and Atezolizumab.

6. The method of claim 1, wherein the at least one immunotherapeutic agent is an antibody selected from the group consisting of siglec 15 antibody, anti-phosphatidylserine, anti-0X40, anti-CD73, anti-TIM3, anti-CD24, anti-CD47, anti-PD1, anti-PDL1, anti-CTLA4, anti-GITR, anti-CD27, anti-CD28, anti-CD122, anti-TIGIT, anti-VISTA, anti-ICOS, and anti-LAG3.

7. The method of claim 1, wherein administering the herbal composition enhances response of the at least one immunotherapeutic agent.

8. The method of claim 1, wherein the herbal composition is administered to the mammal orally.

9. The method of claim 1, wherein administering the herbal composition promotes stimulator of interferon genes (STING) agonist action.

10. The method of claim 8, wherein the herbal composition is administered to the mammal orally in a form selected from the group consisting of a pill, tablet, capsule, soup, tea, concentrate, dragees, liquids, drops, and gelcaps.

11. The method of claim 1, wherein the therapeutically effective amount of the herbal composition is about 20 mg/day to about 2000 mg/day.

12. The method of claim 11, wherein the therapeutically effective amount of the herbal composition is about 1600 mg/day.

13. The method of claim 1, wherein the herbal composition is administered twice daily.

14. The method of claim 1, wherein the herbal composition is administered for about one to about two weeks, followed by a suspension of treatment for at least one week.

15. The method of claim 1, wherein the herbal composition is administered about 30 mins before administering a chemotherapy or a radiation therapy.

16. The method of claim 15, wherein the administering in continued for about 4 days.

17. The method of claim 1, wherein the herbal composition is administered at a time selected from prior to, simultaneously with, and after administration of the one or more immunotherapeutic agent to the mammal.

18. The method of claim 1, wherein the mammal is a human.


SEQUENCE LISTING (28 sequences)

SEQ ID NO: 1 - DNA, 23 bp, Artificial Sequence
  mIFNg Forward
ggcaaaaggatggtgacatgaaa

SEQ ID NO: 2 - DNA, 23 bp, Artificial Sequence
  mIFNg Reverse
tagtaatcaggtgtgattcaatg

SEQ ID NO: 3 - DNA, 21 bp, Artificial Sequence
  mIFNb Forward
tgtctttcttgtcttcagaaa

SEQ ID NO: 4 - DNA, 21 bp, Artificial Sequence
  mIFNb Reverse
tttctgaagacaagaaagaca

SEQ ID NO: 5 - DNA, 23 bp, Artificial Sequence
  mgrB Forward
cactctcgaccctacatggcctt

SEQ ID NO: 6 - DNA, 25 bp, Artificial Sequence
  mgrB Reverse
gtgacatttattatacttccttcac

SEQ ID NO: 7 - DNA, 25 bp, Artificial Sequence
  mperf Forward
gtgtcgcatgtacagttttcgcctg

SEQ ID NO: 8 - DNA, 23 bp, Artificial Sequence
  mperf Reverse
ccgtgataaagtgcgtgccatag

SEQ ID NO: 9 - DNA, 25 bp, Artificial Sequence
  mPDLl Forward
cacttgctacgggcgtttactatca

SEQ ID NO: 10 - DNA, 25 bp, Artificial Sequence
  mPDLI Reverse
gaatcacttgctcatcttccttttc

SEQ ID NO: 11 - DNA, 26 bp, Artificial Sequence
  mPDI Forward
gccaggatggttcttagactccccag

SEQ ID NO: 12 - DNA, 25 bp, Artificial Sequence
  mPDI Reverse
taccagtttagcacgaagctctccg

SEQ ID NO: 13 - DNA, 25 bp, Artificial Sequence
  mIL17a Forward
tcaccctggactctccaccgcaatg

SEQ ID NO: 14 - DNA, 24 bp, Artificial Sequence
  mIL17a Reverse
acagaattcatgtggtggtccagc

SEQ ID NO: 15 - DNA, 21 bp, Artificial Sequence
  mCTLA4 Forward
tcactgctgtttctttgagca

SEQ ID NO: 16 - DNA, 21 bp, Artificial Sequence
  mCTLA4 Reverse
ggctgaaattgcttttcacat

SEQ ID NO: 17 - DNA, 22 bp, Artificial Sequence
  TIMS Forward
ctactacttgcaaggtcattgg

SEQ ID NO: 18 - DNA, 22 bp, Artificial Sequence
  TIMS Reverse
ccaggtgtagatagagtgtaac

SEQ ID NO: 19 - DNA, 23 bp, Artificial Sequence
  ICOS Forward
tcagacttttaacaggagaaatc

SEQ ID NO: 20 - DNA, 23 bp, Artificial Sequence
  ICOS Reverse
tctcagggtatttacaagaaatc

SEQ ID NO: 21 - DNA, 24 bp, Artificial Sequence
  dectinl Forward
agtgctgggtgccctagcattttg

SEQ ID NO: 22 - DNA, 24 bp, Artificial Sequence
  dectinl Reverse
aggctgagaaaaacctcctgtagt

SEQ ID NO: 23 - DNA, 22 bp, Artificial Sequence
  Oasla Forward
gcctttgatgtcctgggtcatg

SEQ ID NO: 24 - DNA, 22 bp, Artificial Sequence
  Oasla Reverse
ccagcttctccttacacagttg

SEQ ID NO: 25 - DNA, 24 bp, Artificial Sequence
  Prkra Forward
gaatcattcatggaaactggaaag

SEQ ID NO: 26 - DNA, 23 bp, Artificial Sequence
  Prkra Reverse
atgtccaaccacgttcgttagag

SEQ ID NO: 27 - DNA, 19 bp, Artificial Sequence
  illSra Forward
ggagaggtatgtctgtaac

SEQ ID NO: 28 - DNA, 22 bp, Artificial Sequence
  illSra Reverse
gaggggtctctgatgcacttga